(19) United States
(12) Patent Application Publication
(10) Pub. No.: US 2012/0311757 A1
(43) Pub. Date: Dec. 13, 2012

(54) LOWER BACK SPINAL SUPPORT

(76) Inventor: James C. Miller, Vauxhall, NJ (US)

(21) Appl. No.: 13/468,857

(22) Filed: May 10, 2012

Related U.S. Application Data
(60) Provisional application No. 61/518,777, filed on May 12, 2011.

(51) Int. Cl. A61F 5/02

(52) U.S. Cl. 2/44

(57) ABSTRACT

The Lower Back Spinal Support is a back support device worn by people laying down all types of flooring to support your upper torso, consisting of a wearable aluminum alloy breast plate padded in a form cushion, with an aluminum cylindrical telescopic swivel leg for height adjustment (which locks into place) and adjustable neck and back straps.

### BACKGROUND OF THE INVENTION

1. Field of the Invention

The present invention is directed to a support device and specifically to a back support device, orthopedic and/or prosthetic, worn to support you're upper body on your knees installing flooring.

2. Description of the Art

Twenty percent of all back injuries come from the work place. Especially at risk for back-related injuries would include the nearly 2,000,000 carpets, floor and tile installers and finishers and professionals in the United States. These types of jobs require the workers to spend a great deal of time on their hands and knees, with their trunk in a full flexed posture often for hours at a time. The static loading of the ligaments and muscle along with the extreme posture of the lumbar spine make this job with potential risk for cumulative trauma. Also, at risk for potential back injuries would be those do-it-yourselfers as many householders attempt to lay down floors for themselves.

### BRIEF DESCRIPTION OF THE DRAWINGS

FIGS. 1-10 are perspective views illustrating a back support, constructed in accordance with the present invention.

### DETAILED DESCRIPTION OF THE PREFERRED EMBODIMENTS

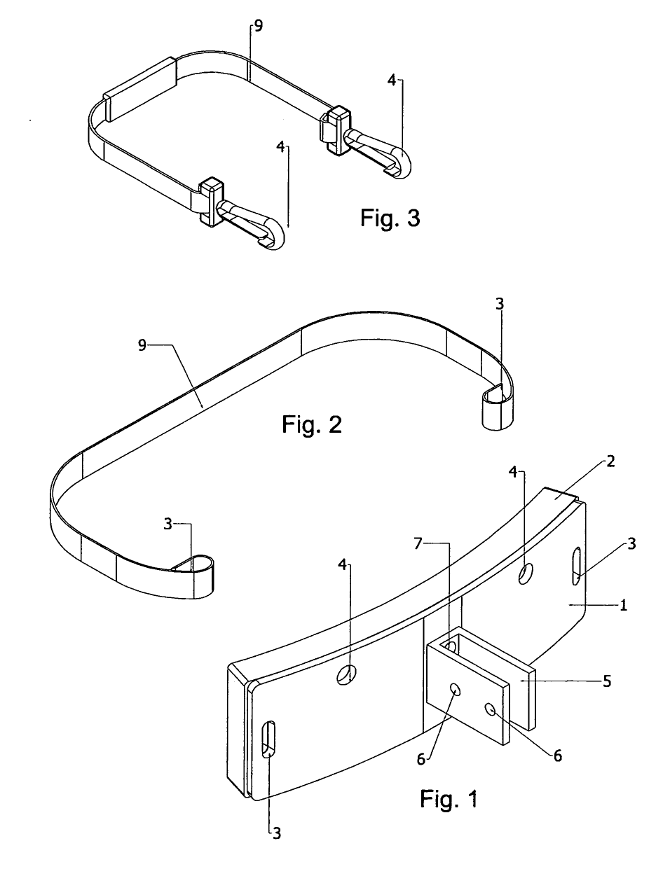

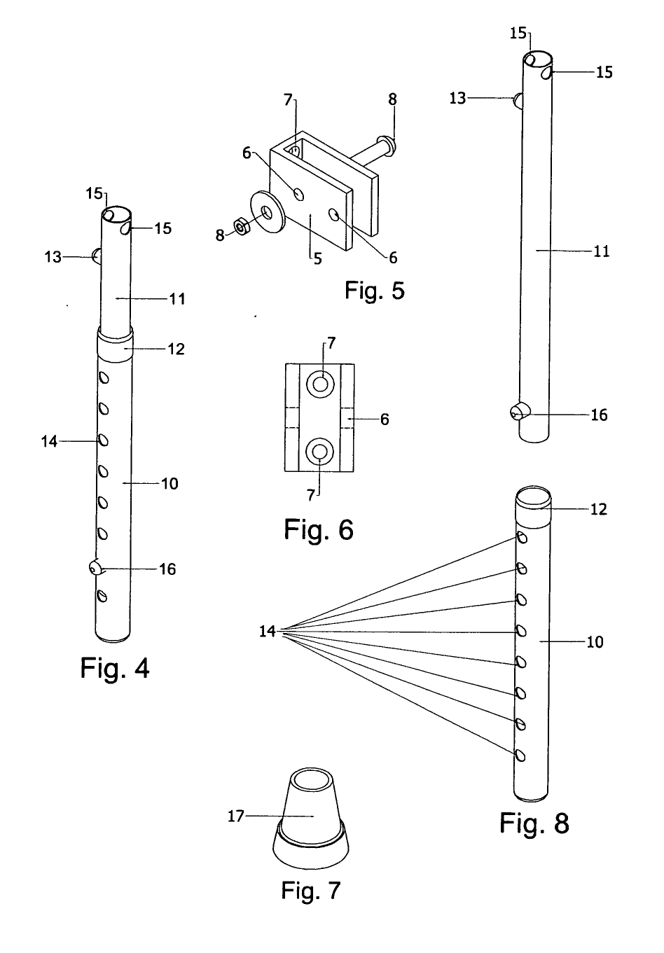

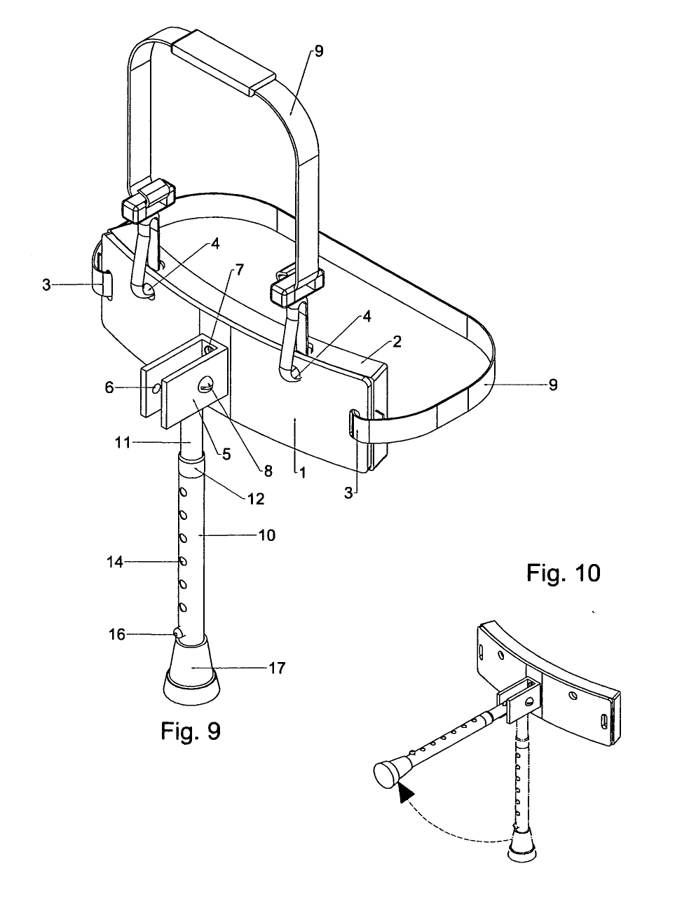

As illustrated in FIGS. 1-10, the present invention is a back support to support the upper body while installing flooring. Enclosed herewith is one cover and (3) sheets of inventor's disclosure hereby incorporated by reference. The back support of the present invention prevents back injuries for people laying down all types of flooring. The back support consists of a wearable breast plate and swivel leg. The breast plate is manufactured in an aluminum alloy measuring 4 inches in height and 13 inches in length. The breast plate is padded in a cushion which would likely be fabricated in a form; also known as NASA form, miracle form, wonder form or viscoelastic form. Developed for space shuttle seating and used for mattresses and a variety of orthopedic products, the form is very dense, and conforms to individual shapes for firm cushion supports. The breast plate is also equipped with adjustable neck and back straps to fit the body. The swivel leg is a cylinder and telescopic for height adjustment, and manufactured in an aluminum alloy for strength and stability extending from the breast plate via a bracket and swivel mechanism. The height of the swivel leg (from chest to floor,) adjust from 13½ inches up to 20½ inches and the shaft terminates in a shock-absorbing and slip-resistant rubber tip; the total weight of the back support is 3 pounds.

Use of the back support of the present invention is simple and straight forward. The user puts the breast plate to their chest adjusting the neck and back straps so that the device fits snugly, next the user lower themselves to their working position on the floor and adjust the height of the swivel leg accordingly. Now the user's upper body is supported which in turn alleviates the pressure and stress on the lower back and spine. When the user gets up, the swivel leg hangs parallel to the user until the next time needed.

The back support of the present invention, a support brace for flooring specialist and do-it-yourselfers, offers distinct and significant benefits and advantages. Foremost the support protects the spine and lower backs of people laying down all types of flooring by supporting their upper torso area. It is hard for anyone to maintain correct posture while on your hands and knees for an extended period of time let alone working all day in that position. After a short time our bodies have difficulty not slumping, which puts pressure on the lower back and spine and eventually can result in injury or worst. The support is of indisputable utility for those who work full-time, part-time, or on occasion laying floors as it provides the necessary upper torso support which is essential in preventing back strains and injuries. Weighing a mere three pounds, the support is fully adjustable, easy to wear, and its unique swivel leg allows the worker to get up and down as often as needed without slowing down their pace. The padded breastplate conforms to the user's body for added comfort and stability, while the swivel leg easily adjust in height to suit all user's. In addition, the swivel leg is equipped with a rubber tip, which creates further stability and support.

The foregoing exemplary descriptions and the illustrative preferred embodiments of the present invention have been explained in the drawings and described in detail, with varying modifications and alternative embodiments being taught. While the invention has been so shown, described and illustrated, it should be understood by those skilled in the art that equivalent changes in form and detail may be made therein without departing from the true spirit and scoop of the invention to be limited only to the claims except as precluded by the prior art. Moreover, the invention as disclosed herein may be suitably practiced in the absence of the specific elements which are disclosed.

1. The Lower back Spinal Support is a device worn to support your upper torso while on your knees installing flooring

2. All invention described and illustrated herein.
